(19) United States
(12) Patent Application Publication
(10) Pub. No.: US 2014/0272803 A1
(43) Pub. Date: Sep. 18, 2014

(54) ROOT CANAL FOR TISSUE IN-GROWTH

(71) Applicant: Alan Nevins, Southampton, NY (US)

(72) Inventor: Alan Nevins, Southampton, NY (US)

(21) Appl. No.: 14/206,820

(22) Filed: Mar. 12, 2014

Related U.S. Application Data
(60) Provisional application No. 61/782,684, filed on Mar. 14, 2013.

(51) Int. Cl. A61K 6/083; A61C 5/04; A61C 5/02

(52) U.S. Cl. CPC A61K 6/0835; A61C 5/023; A61C 5/04
USPC 433/224

(57) ABSTRACT

A composition and method useful in forming a physiologic root canal seal and a method of endontic repair.

### RELATED APPLICATION

The present application claims the benefit of U.S. Provisional Patent Application Ser. No. 61/782,684, filed Mar. 14, 2013, which is herein incorporated by reference in its entirety.

### TECHNICAL FIELD

This invention relates to dental surgery, and more particularly to repairing diseased pulp tissue of the teeth.

### BACKGROUND

When the pulp tissue of a young tooth has been damaged by trauma or caries, to the extent that irreversible pulpitis with significant pulp necrosis is present, the physiological development of the root is compromised and advanced endodontic therapy must be initiated. Traditionally, such routine treatment requires most of the diseased pulp tissue be removed and calcium hydroxide paste or mineral trioxide aggregate cement be placed to allow a thin hard tissue to form and close off the apical portion of the root canal, a procedure known as apexification.

This is done to enhance the placement of the final root canal filling material and prevent extrusion into the periapical tissues. Some tissue growth occurs into the paste or cement within the root canal. As no further hard tissue growth occurs, the treatment leaves the root thin in structure and predisposed to future fracture.

In recent years, there has been a shift in the treatment philosophy of necrotic immature teeth. In some procedures, a blood clot is left in the pulp space to serve as a scaffold for potential periodontal tissue in-growth. These studies showed that under favorable conditions pulp-like tissue could recolonize a pulp-less root canal.

Furthermore, some have demonstrated that pulps with severed circulation could spontaneously repair if the apical foramen was not fully developed. Thus, apically originating tissue, pulp or connective tissue, has the potential to rebuild tissue in the root canal if the apical orifice is large enough to allow tissue in-growth. The necrotic tissue remnants in the pulp space provide a scaffolding effect.

This inherent potential for tissue to proliferate into the necrotic pulp space of an immature tooth is often referred to as “revasculariztion” and is considered to be a regenerative procedure. This tissue growth is also often alternatively termed as “repair,” as the cells and tissue formations normally found in the pulp space of a healthy tooth are not a part of the common tissue repair.

Various apexification and apexogenesis procedures that used calcium hydroxide were performed for many years with variable results. These procedures are time consuming and not fully predictable, mainly due to difficulties to control root canal infection. In response, various disinfection techniques have been suggested to be used in combination with less mechanical debridement. The pulp space with necrotic pulp remnants is typically pre-treated with antiseptic rinses of sodium hypochlorite, with minimal instrumentation, and is dressed with a triple antibiotic paste consisting of ciprofloxacin, metronidazole and amoxicillin (or minocycline). The final coronal seal normally consists of mineral trioxide aggregate and composite resin. This disinfection process is only moderately effective when creating a bacteria-free pulp space that allows for tissue repair and is not sufficient in profoundly infected cases. Animal studies (in dogs) with histopathological evaluations, have shown that treating infected undeveloped teeth with triple antibiotic paste and blood clot technique often result in cementum and bone formation within the canal space. The success rate and predictability of this technique has been only fair, at best, leaving a continuously weak root structure.

The use of collagen has been explored as an alternative. Collagen is the major insoluble fibrous protein that forms the structural basis for all connective tissues and the healing of such. The basic structural unit is a triple-stranded helical molecule composed of three amino acids. Collagen molecules are initially formed within cells, secreted, and then packed together to form long thin fibrils which interdigitate with adjacent fibrils.

Tropo-collagen molecules within a gel derived from solubilized calf skin dermis, when dialyzed with phosphate buffer, have been shown to spontaneously polymerize in-vitro to form collagen fibrils. Such a crystallographic superlattice can serve as a resorbable scaffold that has been demonstrated to be chemotactic for host fibroblasts, supporting cellular migration and attachment. The result of implantation of such a scaffold is an ingrowth of tissue that exhibits a phenotypic expression of both the scaffold's collagen type and host connective tissue at the site of implantation.

However, like apexogenesis and apexification procedures based on calcium hydroxide slurries, the use of collagen thus far has been met with mixed results.

In previous approaches, the use of a collagen gel scaffold resulted in tissue reorganization within pulpectomized root canals and differentiation of this tissue into bone and cementum. However, such a procedure lacked reproducibility due to a dilution of the gel during placement and bacterial contamination occurring prior to and during the procedure. In treatment of one human patient with this technique, complete apexogenesis of a pulpectomized maxillary lateral incisor was observed after six months, with complete of root development and narrowing of the root canal to within normal size of a fully developed adult tooth after one and three years.

In other approaches, collagen is mineralized by combining the collagen fibril assembly and the formation of calcium phosphate in one process step, producing a three-dimensional network of collagen fibrils covered with calcium phosphate. The applied method is useful for studying the mineralization of collagen and offers a promising approach for the development of new bone implant materials. However, as of yet, this material has not been used safely and efficaciously for the repair of teeth.

In other approaches, branching and interconnecting microchannels are incorporated into a solid collagen hydroxypaptite sponge. This design permits the flow of nutrient-rich media with proliferation of new blood vessels to osteogenic cells living deep within the scaffold. The scaffold, therefore, mimics many of the features found in bone. The rationale behind mimicking the compositional and structural organization of the main organic and inorganic components of bone in a single scaffold is twofold. Collagen possesses attractive cell binding properties required for host cells to attach to the scaffold. Carbonate substituted hydroxyapatite provides a source of calcium and phosphate ions that can be used by osteogenic cells to create their own bone. However, as of yet, this material has not been used safely and efficaciously for the repair of teeth.

In still other approaches, a type 1 collagen-hydroxyapatite composite biomaterial enhanced bone formation when implanted into drilled osseous defects, and it was concluded that hydroxyapatite collagen scaffold has a high osteoconductive activity. However, as of yet, this material has not been used safely and efficaciously for the repair of teeth.

Thus, what is needed is an alternative to existing procedures of pulp tissue repair of the tooth that safely and efficaciously promotes in-growth of healthy tissue into the void space of a tooth created during the removal of damaged or diseased pulp tissue. This procedure should provide an environment to promote gradual re-growth of healthy tissue to add to root length and root wall width of the tooth in order to better mimic the structural qualities of the original healthy occurring tissue. The procedure should also seek to reduce infection commonly occurring in existing procedures.

### SUMMARY

This specification describes technologies relating to the repair of damaged or diseased pulp tissue of a human tooth. Implementations of the technology described herein comprise a method of removing diseased pulp tissue from a tooth, disinfecting the tooth, and implanting a collagen matrix coupled with calcium phosphate mineral composite material into the tooth.

Various implementations of the present invention provide benefits that are desirable for dental applications. The procedure results in a stronger biological support so that the tooth is more resistant to fracture or abscess formation. The procedure also promotes gradual regrowth of healthy tissue with an immune system to replace vital pulp tissue that was lost. The procedure thus reduces the possibility of future infection commonly in existing procedures, reducing the potential of unwanted side effects and accelerating recovery time.

In one example implementation, a method of repairing tissue in an immature tooth comprises: accessing a root canal of a tooth by drilling an access opening into the tooth; removing diseased pulp from the root canal; applying a sodium hypochlorite solution to irrigate the canal of the tooth; applying a EDTA and chlorhexidine solution to the root canal; applying a ciprofloxacin-metronidazole paste to the root canal; removing the ciprofloxacin-metronidazole paste from the root canal; molding a filler substance into a shape of the root canal; filling the root canal with the molded filler substance; sealing the access hole with a cement; wherein the filler substance comprises a collagen matrix and calcium phosphate-based mineral composite material.

One or more example implementation may comprise the following features. The sodium hypochlorite solution is approximately 1.0% to 6% sodium hypochlorite. The EDTA and chlorhexidine solution contains about 17% EDTA. The EDTA and chlorhexidine solution contains about 2% chlorhexidine. One to two weeks duration between applying the ciprofloxacin-metronidazole paste and removing the ciprofloxacin-metronidazole paste can be utilized. The calcium phosphate-based mineral is a carbonate apatite. The collagen matrix is a type 1 collagen scaffold. The cements covering the collagen-apatite implant are mineral trioxide aggregate cement or bio-ceramic cement and then covered with glass ionomer cement or composite cement.

The details of one or more implementation of the invention are set forth in the accompanying drawings and the description below. Other features, objects, and advantages of the invention will be apparent from the description and drawings, and from the claims.

### DESCRIPTION OF DRAWINGS

FIG. 1 is a schematic view of a human tooth and its surrounding tissue.

FIG. 2 shows the drilling of an access opening into a tooth.

FIG. 3 shows the removal of pulp tissue from a tooth using various files.

FIG. 4 shows a tooth with its pulp tissue removed.

FIG. 5 shows the application of a disinfectant solution.

FIG. 6 shows the molding of a filling substance before insertion into a tooth.

FIG. 7 shows a filling substance inserted into a tooth.

FIG. 8 shows closing an access channel of the tooth with a sealing agent.

FIG. 9 shows a sealed tooth.

### DETAILED DESCRIPTION

As shown in FIG. 1, a human tooth 100 has a pulp cavity 102. Pulp cavity 102 is typically filled with pulp tissue 104, which includes nerve and blood vessels that serve to connect the tooth to the nervous system and circulatory system, respectively.

When the pulp tissue 104 becomes damaged, for example due to trauma or disease, damaged pulp tissue 104 can be partially or totally removed, and the tooth allowed to heal through in-growth of healthy tissue and subsequent recalcification. This can be performed through an improved “root canal” procedure. As shown in FIG. 2, in an example implementation of a root canal procedure, an operator begins by drilling an access channel 202 through the crown 204 and neck 206 of tooth 100 and into root 210, creating a path to pulp cavity 102. Drilling can be performed using any device common to the field of dental surgery, such as a dental drill 208.

As shown in FIG. 3, after access channel 202 is created, diseased pulp tissue 104 is removed from pulp cavity 102 through access channel 202 using various debridement instruments. Debridement instruments can include various instruments common to the field of dental surgery. As examples, debridement instruments can include instruments with sharpened awl-like operating surfaces (e.g., instrument 302a) for incising and separating tissue from tooth 100, instruments with scoop-like operating surfaces (e.g., instrument 302b) for coarse removal of tissue, or instruments with file-like operating surfaces (e.g., instrument 302c) for fine removal of tissue. Debridement tools, such as instruments 302a-c, can be operated by hand, or through a motorized mechanism.

As shown in FIG. 4, after debridement, pulp cavity 102 is clear of tissue. An additional void space 402 can also be created using debridement instruments 302. Void space 402 extends beyond root 210 of tooth 100 and into the surrounding tissue 404.

As shown in FIG. 5, the operator then disinfects the exposed surfaces of tooth 100. These surfaces include the exterior portion of tooth 100, as well as the inner portions of tooth 100 that were exposed during debridement. This can include, for example, the surfaces of pulp cavity 102 and void space 402. During disinfection, the operator applies a disinfectant solution 502 to directly contact these surfaces. As shown in FIG. 5, to ensure that disinfectant solution 502 thoroughly contacts all exposed surfaces of tooth 100, a syringe device 504 can be used to apply disinfectant solution 502 into relatively enclosed areas, such as pulp cavity 102 and void space 402.

In some implementation, disinfectant solution can be a sodium hypochlorite solution. For example, a solution of 1.0% to 6% sodium hypochlorite can be used. In some implementations, an ethylenediaminetetraacetic acid (EDTA) and chlorhexidine solution can be used. For example, a solution containing about 17% EDTA and 2% chlorhexidine can be used. Multiple disinfectant solutions can be used in place of or in conjunction with other disinfectant solutions. In some implementations, some disinfectant solutions can be used for one portion of tooth 100, for instance only on the exterior surfaces, while other disinfectant solution can be used for other portions of tooth 100, for instance in pulp cavity 102 and void space 402.

The operator can further disinfect tooth 100 by applying a disinfectant paste into pulp cavity 102 and void space 402. In some implementations, the disinfectant paste is a ciprofloxacin-metronidazole paste. In these implementations, am operator fills pulp cavity 102 and void space 402 with ciprofloxacin-metronidazole paste, then seals access channel 202 with a sealing agent, such a dental cement. After a period of approximately one to two weeks, access channel 202 is reopened and the disinfectant paste is removed. The removal of disinfectant paste can be the same or similar to the debridement process described above.

To allow the pulp tissue, periodontal tissue or bone tissue (or a combination of these tissues) to regrow into the void created during the debridement process, a filler substance 602 can be used to fill the voids in pulp cavity 102 and void space 402. Filler substance 602 is of a moldable, putty-like consistency, and is osteoconductive, such that cells may grow into and within filler substance 602.

In some implementations, filler substance 602 is a collagen matrix and calcium based mineral composite material. The calcium based mineral can be of various compositions. For instance, in some implementations, it can be predominantly a carbonate apatite. In some implementations, it can be carbonate apatite, calcium phosphate, calcium sulfate, calcium carbonate, an organic bone mineral, or a combination of these substances. The calcium-based mineral can have particle sizes of roughly 25-2000 μm. The calcium-based mineral can be derived from synthetic or naturally-occurring sources.

The collagen matrix can be of various compositions. For instance, it can be composed of type 1 collagen fibers, cross-linked to create a three-dimensional matrix. This matrix can vary in arrangement to create a matrix with varying pore sizes, density, tensile strength, or other physical characteristics. For instance, the cross-linking density of the collagen fibers can be varied to provide a matrix with varying pore sizes. In some implementations, the cross-linking density can be selected to produce a matrix with a pore size of approximately 50-500 μm.

Filler substance 602 can include varying proportions of collagen and calcium-based mineral in order have particular porosity, density, tensile strength, compression strength, or other physical characteristics. In some implementations, filler substance 602 can include approximately 3-60% collagen and 40-97% calcium-based mineral by weight. In some implementations, filler substance 602 can be anhydrous, and may be rehydrated prior to use.

Filler substance 602 is osteoconductive, such that cells can grow into and within filler substance 602. The collagen matrix allows substantial cellular ingrowth, and acts as both a structural support and a guide for the growth of tissue.

In some implementations, substance 602 can be very viscous, such that it holds its shape after insertion into pulp cavity 102 or void space 402.

As shown in FIG. 6, filler substance 602 is rehydrated, if necessary, molded into the general shape of pulp cavity 102 and void space 402, and inserted into tooth 100. As shown in FIG. 7, upon insertion into tooth 100, filler substance 602 fills the entity of pulp cavity 102 and void space 402, leaving no air gaps between filler substance 602 and the surrounding tissue 404.

As shown in FIG. 8, access channel 202 is re-closed with dental cement 802. Cement 802 can be a mineral trioxide aggregate cement, bio-ceramic cement, a glass ionomer cement, or composite cement.

Initially, tooth 100 is structurally supported predominantly by the calcium-mineral materials of filler substance 602. However, as shown in FIG. 9, over time surrounding tissue 404 begins to regrow. As filler substance is 602 is osteoconductive, cellular growth from both the surrounding tissue 404 and tooth 100 can extend into the scaffold-like matrix filler substance 602 and re-calcify tooth 100. This allows tooth 100 to regain its structural strength from naturally growing tissue, utilizing a three-dimensional scaffold to guide and support the ongoing tissue growth. After tissue is fully regrown into filler substance 602, the calcium-mineral materials of filler substance 602 continue to provide rigidity and strength to tooth 100.

During this process, the operator can periodically or continuously monitor the process using various imaging modalities, such as using x-ray images (including cone beam CT scan) or fluoroscopy. To improve the visibility of the tools and substances associated with this procedure during the imaging, each instrument or substance can include imaging contrast agents to provide imaging contrast. Materials may include radiopaque materials, such that they are provide imaging contrast during x-ray or fluoroscopic procedures, paramagnetic or super paramagnetic materials, such that they provide imaging contrast during MRI, or other contrast agents commonly used with other imaging modalities. As an example, in some implementations, filling substance 602 can include contrast agents.

While an example root canal procedure is described above, modifications can be made to this example procedure, depending on the implementation. For instance, in some implementations, instead of accessing pulp cavity 102 through the crown to the tooth 100, pulp cavity 102 can be accessed, either partially or entirely, through another portion of the tooth. As an example, a portion of substance 602 can be extruded through the apical foramen or apex of tooth 100. This may be preferable in certain circumstances, for example when it is preferable that substance 602 initially makes contact with bone and periodontal ligament cells. Substance 602 can also be introduced through other parts of the tooth, depending on the implementation. In some implementations, it is not necessary to dry the pulp cavity 102 and void space 402 prior to introduction and placement of substance 602.

Substance 602 can be composed of various of materials, and can vary depending on the implementation. In general, substance 602 can be a collagen-based material, for instance a collagen gel scaffold as described above. Collagen is the major insoluble fibrous protein that forms the structural basis for all connective tissues and the healing of such. The basic structural unit is a triple-stranded helical molecule composed of three amino acids. Collagen molecules are initially formed within cells, secreted, and then packed together to form long thin fibrils which interdigitate with adjacent fibrils.

Tropo-collagen molecules within a gel derived from solubilized calf skin dermis, when dialyzed with phosphate buffer, have been shown to spontaneously polymerize in-vitro to form collagen fibrils. Such a crystallographic superlattice can serve as a resorbable scaffold that has been demonstrated to be chemotactic for host fibroblasts, supporting cellular migration and attachment. The result of implantation of such a scaffold is an ingrowth of tissue that exhibits a phenotypic expression of both the scaffold's collagen type and host connective tissue at the site of implantation.

Further, collagen has the ideal property of being hemostatic, which results in the incorporation of fibrin and growth factors within the scaffold. These substances have the potential to enhance the healing of new tissue within the canal space.

Further still, collagen as a scaffolding material can provide enhanced predictability over calcium hydroxide slurry for repair of pulp space tissue. Thus, success in tissue regeneration can be enhanced with increase in collagen density and cross linking.

Initial studies conducted in young teeth of Rhesus monkeys using a collagen gel scaffold, calfskin collagen at 10 mg/ml, resulted in tissue reorganization within pulpectomzed root canals and differentiation of this tissue into bone and cementum. It was concluded that some lack of reproducibility was due to a dilution of the gel during placement and bacterial contamination occurring during the procedure. A similar but more concentrated preparation (20 mg/ml) was used to affect complete apexogenesis of a pulpectomized maxillary lateral incisor of a child who had experienced a traumatic injury. This first human case report was published with 6 month results only. One and three year follow-up radiographs demonstrated completion of root development and narrowing of the root canal to within normal size of a fully developed adult tooth.

Further studies with cross-linked versions of collagen gel rendered more reproducible results in teeth that were not previously infected and moderately successful results in teeth that had previous infections. Positive results of these studies were reproduced using cross-linked collagen sponge. Commercially available injectable collagen (e.g., Zyplast®), has been used in a clinical trial, and has been evaluated in guinea pigs. Results showed that Zyplast® was well tolerated and seamlessly replaced by bone. Further, Zyplast has a highest density of collagen (e.g., 65 mg/ml), is cross linked, and is homogenized, which makes it syringable. Accordingly, in some implementations, substance 602 can include Zyplast®.

In some implementations, substance 602 can include a buffered solution of colloidal collagen, a calcium salt, a phosphate salt and Lugol's solution (for example as described in U.S. Pat. No. 3,968,567, incorporated herein by reference). This composition can be useful, for example, in forming a physiologic root end.

In some implementations, substance 602 can include SynOss (manufactured by Collagen Matrix/Dental, a division of Collagen Matrix, Inc., Oakland, N.J.). S SynOss is a bovine derived type 1 collagen-hydroxyapatite sponge which is comparable chemically to the mineralized matrix of human bone. This material is similar chemically to the cross linked collagen calcium phosphate gel as described above. This material, when implanted into infrabony defects, resorbs and is replaced by new bone as part of the healing process. It is chemotactic for osteoblasts and stem cells, supporting cellular migration and attachment. Because SynOss is approved by the Federal Food and Drug Administration for use in dentistry, clinical implementations of the above described procedure can include the use of SynOss as substance 602. In some implementations, the use of SynOss can provide enhanced predictability over calcium hydroxide slurry, MTA placement or blood clot technique (revascularization) to effect hard tissue growth into the root canals of immature teeth. The constraints of the presence of a previous failing conventional treatment, narrow canal and apical foramen size, and presence of periapical pathology do not appear to obviate the possibility of successful revitalization. This bioactive scaffold material is easy to place and is compatible in technique with the presently suggested revascularization protocol. In an example implementation, SynOss can be used to regenerate calcified tissue within young pulpless or partially pulpless teeth in humans.

In some implementations, substance 602 can be used in conjunction with stem cells to regenerate the pulp-dentin complex progress. An appropriate substance 602, for example SynOss, could provide a scaffold construct for such implementations.

To demonstrate the effectiveness of the above implementations, several case studies are described below.

In a first example, a 48 year old female patient was evaluated for a chief complaint of pain and moderate swelling on the right side of her mandible related to an acute abscess of tooth #29. Moderate swelling of the buccal mucosa was evident. A large composite filling was in place. Clinical examination revealed the tooth to be sensitive to percussion and to palpation of the buccal mucosa. Radiographs demonstrated a peripical radiolucency to be present approximately 7 mm in diameter. Root canal treatment had been performed several years prior to this examination. Retreatment with regenerative endodontic treatment was selected by the patient after various options were reviewed with her. Informed consent was reviewed and signed.

Endodontic access preparation using rubber dam isolation was performed after local anesthetic was given which consisted of a combination of 1.7 mL Xylocalne 2% with epinephrine 1:100,000 (Dentsply, Cambridge, Ontario) and 1.7 mL Septocaine 4% with epinephrine 1:100,000 (Septodent, Louisville, Colo.).

The existing gutta percha root canal filling was removed and working length determined radiographically with a size 60 K-file. The canal was instrumented, irrigated with 6% sodium hypochlorite 10 cc (Coltene/Whaledent, Cuyahoga Falls, Ohio) and 17% EDTA 10 cc (Coltene/Whaledent, Cuyahoga Falls, Ohio) and dried with paper points. A cream-like consistency of ciprofloxacin and metronidazole mixed in equal amounts was placed into the canal using a lentulo spiral to a point 1 mm short of the root apex. Cavit (3M ESPE, St Paul, Minn.) filling was placed into the access opening.

On a subsequent visit, the tooth was re-opened and irrigated with 2% chlorhexidine gluconate 10 cc (Coltene/Whaledent, Cuyahoga Falls, Ohio) dried with paper points and bleeding into the canal induced with an endodontic file. SynOss (Deputy Spine, Raynham, Mass.) implant material was soaked in normal saline solution, cut into several pieces and placed into the canal.

Some material was placed through the apical foramen into periapical tissue and the rest used to fill the entire volume of the canal to the cervical region. MTA cement (Dentsply, Tulsa, Okla.) was used to cover the SynOss and a moist cotton pellet, moistened with saline, was placed over the MTA. Cavit was used to cover the cotton.

In a subsequent visit, the cotton was removed and composite filling placed to cover the MTA. Recall visits were scheduled at 3 month intervals for a period of one year.

At the 3 month recall examination the patient was asymptomatic clinically and radiographs showed partial healing of the periapical radiolucency. A small amount of root canal filling material that had been extruded into the periapical tissue during instrumentation appeared to have migrated toward the apex of the tooth. Diffuse radio-opacity was developing throughout the canal space, indicating formation of bone and cementum root structure within the canal space.

At the 6 month recall examination the patient remained asymptomatic. The periapical lesion appeared to continue healing. The gutta percha fragment was resorbing and continued to migrate further toward the apex of the tooth. The canal space continued to become more radio-opaque.

In another example, A 14 year old female patient was referred for evaluation of tooth #13 by her orthodontist and general dentist. She was asymptomatic and had a fixed orthodontic appliance in place. Tooth #13 had an accessory cusp on the palatal which was fractured and caries on the occlusal surface. Tooth #13 was negative to percussion, palpation, biting, the electric pulp test and cold testing. Periodontal probing was within normal limits and the tooth had no mobility since an orthodontic appliance was in place. Radiographs (Carestream, Atlanta, Ga.) demonstrated a periapical radiolucency to be present approximately 3 mm in diameter and the tooth had an open apex. Cone beam computed tomography was performed (Carestream 9000, Atlanta, Ga.) and the open apex was measured to be approximately 1.5 mm. Treatment with regenerative endodontics was selected by the patient and her parents after various options were reviewed with them. Informed consent was reviewed and signed.

The patient was given a buccal and palatal infiltration of 3.4 mL Septocaine 4% with epinephrine 1:100,000 (Septodont, Louisville, Colo.). A rubber dam was placed and the fractured accessory cusp was removed, caries excavated, and endodontic access achieved. The canal was gently debrided with endodontic files and working length determined radiographically with a size 70 hedstrom file. The canal was instrumented, irrigated with 6% sodium hypochlorite 10 cc (Coltene/Whaledent, Cuyahoga Falls, Ohio) using an Endovac (Discus Dental, Culver City, Calif.) and dried with paper points. A cream-like consistency of ciprofloxacin and metronidazole mixed in equal amounts was placed into the canal. Cavit (3M ESPE, St Paul, Minn.) filling was placed into the access opening.

One month later the patient returned and was given a buccal infiltration of 1.7 ml of Mepivicane plain (Septodont Louisville, Colo.). The tooth was re-opened and irrigated with 17% EDTA 20 cc (Henry Schein, Melville, N.Y.) dried with paper points and bleeding into the canal induced with an endodontic file.

SynOss (Deputy Spine, Raynham, Mass.) implant material was soaked in normal saline solution, cut into several pieces and placed into the canal. Some material was placed through the apical foramen into periapical tissue and the rest used to fill the entire volume of the canal to the cervical region. BC Putty (Brasseler USA, Savannah, Ga.) was used to cover the SynOss and a moist cotton pellet, moistened with saline, was placed over the BC Putty. Cavit was used to cover the cotton.

One week later, the Cavit and cotton were removed, a glass ionomer base was placed and composite filling placed to cover the BC Putty.

At the 6 month recall examination the patient remained asymptomatic.

Radiographic examination revealed that the periapical lesion was resolving. The coronal two thirds of the canal space was becoming radiopaque and there was evidence of apical closure.

The above examples demonstrate that healing of the infrabony periapical lesion and continued development of the root apex is evident within approximately 6 months of treatment. Migration of the residual gutta percha in the retreatment case is likely a result of progenitor cells and macrophages migrating toward the root canal space, carrying the gutta percha with it.

The rapid and complete hard tissue formation within the canal space in each of the above examples is significant from the prospective of minimizing future possibility of root fracture. A close adaptation of new hard tissue to existing root canal dentin appears to be present as shown in cone beam images, suggesting a chemical bond between the two. New tissue likely contains soft tissue inclusions and blood vessels that normally exist in new bone formation, represented by radiolucent spaces within this tissue. Introduction of an immune system into canals that were previously infected would probably reduce the possibility of residual recurrent microbial infection.

Although new tissue in-growth is probably bone and cementum, rather than pulp with dentin, the above examples are compatible with clinical requirements to better maintain the function of teeth. Avulsed and re-implanted immature teeth have a similar bone formation within the canal, which is thought to be a result of remnants of devascularized pulp, consisting mostly of collagen fibers, serving as a scaffold.

With better disinfection protocols presently in use and a collagen-hydroxyapatite scaffold material that has been refined and used successfully for implantation in bone, results in previously infected teeth with apical periodontitis appear to be reproducible and consistent.

A number of implementations have been described. Nevertheless, it will be understood that various modifications may be made without departing from the spirit and scope of the invention. Accordingly, other embodiments are within the scope of the following claims.

What is claimed is:

1. A method of repairing tissue in an immature tooth comprising:
accessing a root canal of a tooth by drilling an access opening in the tooth;
removing diseased pulp from the root canal;
applying a sodium hypochlorite solution to the root canal and an antiseptic to the exterior surface of the tooth;
applying a EDTA and chlorhexidine solution to the root canal applying a ciprofloxacin-metronidazole paste to the root canal;
removing the ciprofloxacin-metronidazole paste from the root canal;
molding a filler substance into a shape of the root canal;
filling the root canal with the molded filler substance;
sealing the access hole with a cement;
wherein the filler substance comprises a collagen matrix and calcium-based mineral composite material.

2. The method of claim 1, wherein the sodium hypochlorite solution is approximately 1.0% to 6.0% sodium hypochlorite.

3. The method of claim 1, wherein the EDTA and chlorhexidine solution contains about 17% EDTA.

4. The method of claim 1, wherein the EDTA and chlorhexidine solution contains about 2% chlorhexidine.

5. The method of claim 1, further comprising waiting about one to two weeks between applying the ciprofloxacin-metronidazole paste and removing the ciprofloxacin-metronidazole paste.

6. The method of claim 1, wherein the calcium-based mineral is a carbonate apatite.

7. The method of claim 1, wherein the collagen matrix is a type 1 collagen scaffold.

8. The method of claim 1, wherein the cement is mineral trioxide aggregate cement, bio ceramic cement, glass ionomer cement, or composite cement.
